(19) United States
(12) Patent Application Publication
(10) Pub. No.: US 2024/0085185 A1
(43) Pub. Date: Mar. 14, 2024

(54) SUBMERSION DETECTION, UNDERWATER DEPTH AND LOW-LATENCY TEMPERATURE ESTIMATION USING WEARABLE DEVICE

(71) Applicant: Apple Inc., Cupertino, CA (US)

(72) Inventors: Stephen P. Jackson, San Francisco, CA (US); Ti-Yen Lan, Palo Alto, CA (US); Yi Wen Liao, San Jose, CA (US); Alexandru Popovici, Santa Clara, CA (US); Igor Tchertkov, Los Gatos, CA (US); Rose M. Wahlin, San Francisco, CA (US); Natisa Jeyakanthan, Mountain View, CA (US); Amit K. Jain, Fremont, CA (US); Kenneth M. Lee, Morgan Hill, CA (US)

(21) Appl. No.: 18/462,324

(22) Filed: Sep. 6, 2023

Related U.S. Application Data
(60) Provisional application No. 63/436,450, filed on Dec. 30, 2022.
(60) Provisional application No. 63/404,154, filed on Sep. 6, 2022.

(51) Int. Cl. G01C 21/16

(52) U.S. Cl. CPC G01C 21/165; G01C 21/183

(57) ABSTRACT

Embodiments are disclosed for submersion detection and underwater depth and low-latency temperature estimation. In an embodiment, a method comprises: determining a first set of vertical accelerations obtained from an inertial sensor of a wearable device; determining a second set of vertical accelerations obtained from pressure data; determining a first feature associated with a correlation between the first and second sets of vertical accelerations; and determining that the wearable device is submerged or not submerged in water based on a machine learning model applied to the first feature. In another embodiment, a method comprises: determining a submersion state of a wearable device; and responsive to the submersion state being submerged, computing a forward estimate of water temperature based on measured ambient water temperature at the water surface, a temperature error lookup table, and a rate of change of the ambient water temperature.

### TECHNICAL FIELD

This disclosure relates generally to submersion detection and underwater depth and temperature estimation.

### BACKGROUND

Users participating in recreational underwater activities, such as scuba diving, snorkeling, underwater pool swims and shallow free diving, can benefit from various underwater sensing, including current depth, maximum depth, time under water and water temperature. Such sensing typically requires specific underwater sensing devices, such as wrist-worn dive computers and/or bulky mechanical gauges. Such devices provide little or no benefit to the user for land-based activities. Accordingly, there is a need for a single wearable device that can provide environment sensing for underwater and land-based recreational activities.

### SUMMARY

Embodiments are disclosed for submersion detection and underwater depth and low-latency temperature estimation.

In some embodiments, a method comprises: determining, with at least one processor, a first set of vertical accelerations obtained from an inertial sensor of a wearable device; determining, with the at least one processor, a second set of vertical accelerations obtained from pressure data; determining, with the at least one processor, a first feature associated with a correlation between the first and second sets of vertical accelerations; and determining, with the at least one processor, whether the wearable device is submerged or not submerged in water based on a machine learning model applied to the first feature.

In some embodiments, the method further comprises determining, with the at least one processor; a second feature associated with a slope of a line fitted to a plot of the first set of accelerations and the second set of accelerations; and determining, with the at least one processor, that the wearable device is submerged or not submerged in the water based on a machine learning model applied to the first feature and the second feature.

In some embodiments, the method further comprises determining, with the at least one processor, a second feature associated with a touch screen gesture; and determining, with the at least one processor, whether the wearable device is submerged or not submerged in the water based on a machine learning model applied to the first feature and the second feature.

In some embodiments, responsive to determining that the wearable device is submerged in water, the method further comprises estimating, with the at least one processor, a depth of the wearable device based on a measured pressure and a stored measured air pressure computed and stored by the wearable device prior to the wearable device being submerged in the water.

In some embodiments, determining whether the wearable device is submerged or not submerged in the water further comprises comparing the estimated depth with a minimum depth threshold.

In some embodiments, a method comprises: determining, with at least one processor, a water submersion state of a wearable device; and responsive to the submersion state being submerged, computing, with the at least one processor, a forward estimate of the water temperature based on a measured ambient water temperature, a temperature error lookup table, and a rate of change of the ambient water temperature.

In some embodiments, the ambient air pressure at the surface is measured each time the first set of vertical accelerations are above a minimum threshold and a range of measured pressure change is less than or equal to a specified pressure threshold.

Other embodiments are directed to an apparatus, system and computer-readable medium.

Particular embodiments described herein provide one or more of the following advantages. Upon detection that the wearable device is submerged, water temperature is forward estimated using a heat transfer model, thus avoiding the user having to hold their body in an uncomfortable position (e.g., the hand submerged in cold water) while the embedded temperature sensor reaches thermal equilibrium with the water. Ambient air pressure above the water surface is periodically measured and stored on the wearable device in response to trigger events based on wearable device motion (e.g., inertial vertical acceleration) or radio signal reception on the wearable device. A submerged/de-submerged classifier detects when the wearable device is submerged/de-submerged based on features derived by comparing vertical acceleration from an inertial measurement unit (IMU) with vertical acceleration computed from pressure data and other signals (e.g., palm gesture detection, electrocardiogram (ECG) electrode short detection). Responsive to detecting that the wearable device is submerged, the underwater depth is computed by the wearable device using pressure data measured underwater (e.g., by an embedded barometer) and the stored ambient air pressure above the water surface.

### BRIEF DESCRIPTION OF THE DRAWINGS

FIG. 1 is an example wearable device for submersion detection, underwater depth estimation and low-latency temperature estimation, according to one or more embodiments.

FIG. 2 is a system block diagram of low-latency water temperature estimation, according to one or more embodiments.

FIG. 3A is a graph illustrating low-latency temperature estimation, according to one or more embodiments.

FIGS. 3B and 3C illustrate an example thermal model, according to one or more embodiments.

FIG. 4A illustrates the difference between atmospheric pressure at the surface of water as a function of elevation, according to one or more embodiments.

FIG. 4B is a graph illustrating motion-based surface pressure calibration on wrist raise, according to one or more embodiments.

FIG. 4C illustrates radio-based surface pressure calibration, according one or more embodiments.

FIG. 5A is a block diagram of a system for submersion detection and underwater depth estimation, according to one or more embodiments.

FIG. 5B is a block diagram of the surface pressure calibration system of FIG. 5A, according to one or more embodiments.

FIG. 6A is a graph of depth versus time before and after submersion, according to one or more embodiments.

FIG. 6B is a graph of vertical acceleration versus time showing the tracking of filtered acceleration with differentiated pressure before and after submersion, according to one or more embodiments.

FIG. 7A is a graph of vertical acceleration from pressure versus vertical acceleration from IMU, illustrating the correlation between IMU vertical acceleration and vertical acceleration derived from pressure data, according to one or more embodiments.

FIG. 7B illustrates machine learning to recognize submersion from other pressure disturbances that could cause false positives, according to one or more embodiments.

FIG. 8 is a flow diagram of a process for temperature estimation, as described in reference to FIGS. 1-7.

FIG. 9 is a flow diagram of a process for submerged/de-submerged detection, as described in reference to FIGS. 1-7.

FIG. 10 is a flow diagram of a process for depth estimation, as described in reference to FIGS. 1-7.

FIG. 11 is a block diagram of a wearable device architecture for implementing the features and processes described in reference to FIGS. 1-10.

### DETAILED DESCRIPTION

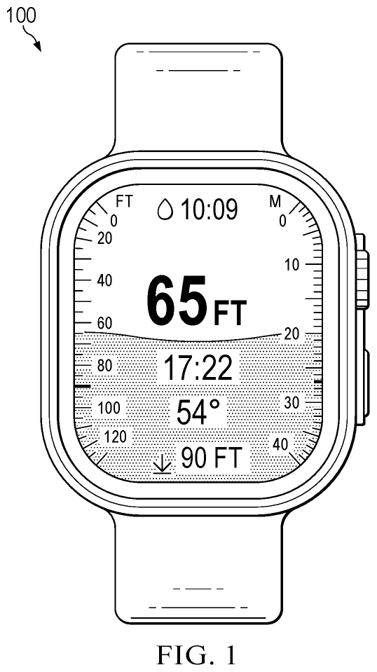

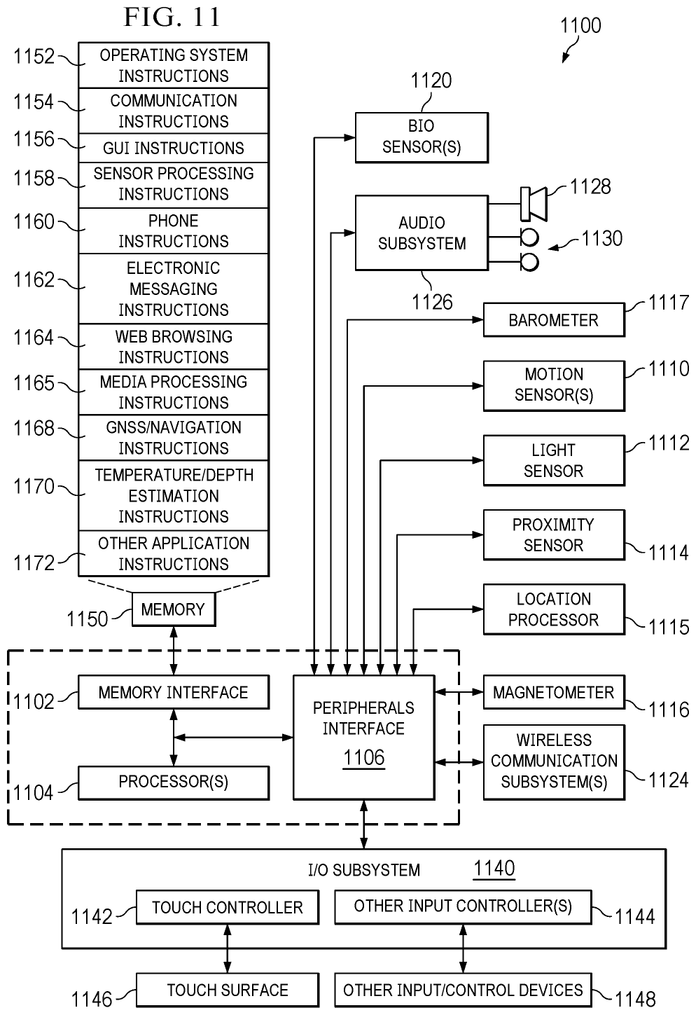

FIG. 1 is an example wearable device for submersion detection, underwater depth estimation and low-latency temperature estimation, according to one or more embodiments. In the example shown, wearable device 100 is a smartwatch having an example architecture as described in reference to FIG. 11. Other wearable devices can include but are not limited to wearable dive computers or any other wearable device suitable for underwater use.

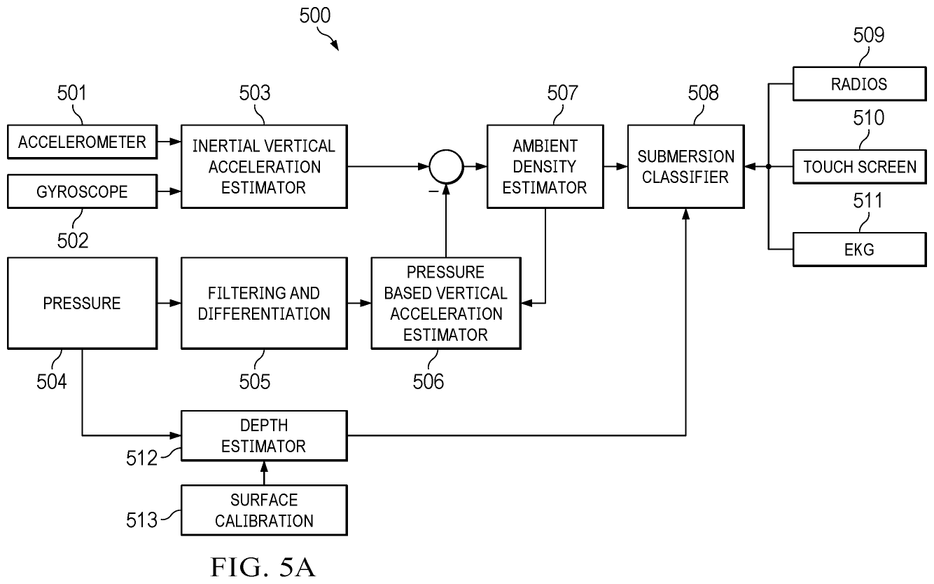

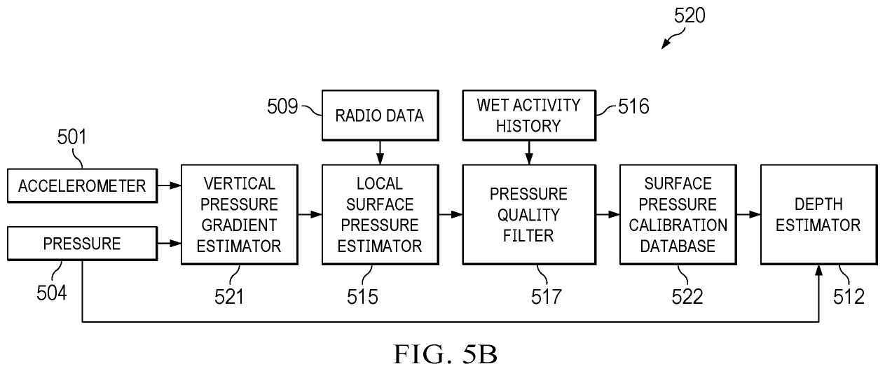

In some embodiments, wearable device 100 communicates wirelessly with a companion device (e.g., a smartphone) through, for example, a short-range communication link (e.g., paired with a smartphone using Bluetooth). Radio frequency (RF) signals sent from the companion device to wearable device 100 can be monitored by wearable device 100 to detect submerged/de-submerged events, as described more fully in reference to FIG. 5A.

During recreational underwater activities such as scuba diving, snorkeling, underwater pool swims, and shallow free diving, a depth application running on wearable device 100 can show the time, depth, water temperature, and the session's maximum depth while the user has been underwater. Other applications include, e.g., a dive planning computer that provides, e.g., depth, maximum depth, compass heading, dive time, water temperature and safety warnings (e.g., safety stops, ascent rate, max depth, cold). In some implementations, wearable device 100 includes a haptic engine to provide force feedback to the user regarding, e.g., safety warnings.

### Low-Latency Water Temperature Estimation

Temperature sensors in existing wearable devices are delayed in reaching thermal equilibrium with water because they are embedded in the thermal mass of the device. Temperature estimation with these wearable devices often requires the user to hold their body in an uncomfortable position or at an uncomfortable temperature (e.g., submerged in cold underwater) for an extended period of time.

Wearable device 100 addresses these issues by modeling the thermodynamics of the heat transfer between the water, wearable device 100 and the user to obtain a heat transfer rate. The heat transfer rate is then used to estimate the water temperature before wearable device 100 has come to thermal equilibrium with the water. In some embodiments, multiple sensors in wearable device 100 can be used to improve temperature estimation and compensate for any heat generation with wearable device 100. Multiple sensors in wearable device 100 can be used to detect unmodeled thermal disturbances and improve temperature uncertainty estimates. In some embodiments, submersion detection can be used to select and start the appropriate thermodynamic model.

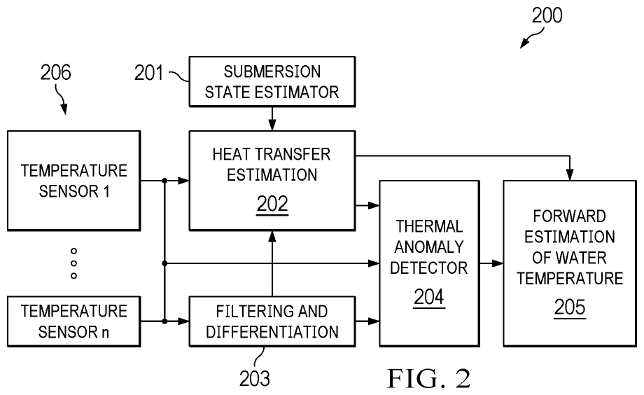

FIG. 2 is a system block diagram of low-latency water temperature estimation in wearable device 100, according to one or more embodiments. System 200 includes submersion state estimator 201, heat transfer estimator 202, filtering and differentiation 203, thermal anomaly detector 204, water temperature estimator 205 and temperature sensors 206.

Submersion state estimator 201 selects a thermal model based on whether the wearable device is submerged in water or not submerged in water, as described more fully in reference to FIG. 5A. The thermal model is input into heat transfer estimator 202 together with temperature data from multiple temperature sensors 206 embedded in wearable device 100, and a predicted rate of change of temperature computed by differentiating temperature. Filtering and differentiation 203 filters and decimates the temperature data from temperature sensors 206 to remove potential outliers in the temperature data.

Thermal anomaly detector 204 receives the heat transfer estimate, heat transfer rate and temperature sensor data and computes a rate of change of temperature. Thermal anomaly detector 204 compares the heat transfer rate with the rate of change of temperature and determines if any of the temperature sensors are not converging in a predictable manner to the same temperature due to external thermal disturbances (e.g., user's body temperature, sunlight) and/or internal thermal disturbances (e.g., system-on-chip (SoC) heating up, light emitting diodes (LED) sensors used for heart rate sensing heating up). Thermal anomaly detector 204 also computes an uncertainty value for the water temperature estimation. For example, if thermal anomalies are detected, by thermal anomaly detector 204, the water temperature estimate uncertainty will increase and vice-versa.

Water temperature estimator 205 takes the heat transfer rate and uncertainty and outputs the estimated water temperature and corresponding uncertainty value to applications running on the wearable device (e.g., diving applications).

In some embodiments, a first order forward temperature predictor is given by:

\hat{T}_{water}=T(t)−ΔT(\dot{T}(t)) [1]

where \hat{T}_{water} is the estimated water temperature, T(t) is the measured external temperature at time t, ΔT is a temperature error from a pre-established lookup table, which is a function of \dot{T} and \dot{T}(t) (Celsius (C)/second) is the rate of change of temperature at time t, which is computed by differentiating the measured external temperature T(t). In some embodiments, a real-time, low-pass filtered estimate of \dot{T}(t) is computed. In other embodiments, \dot{T}(t) can be determined from a buffer of past temperature measurements.

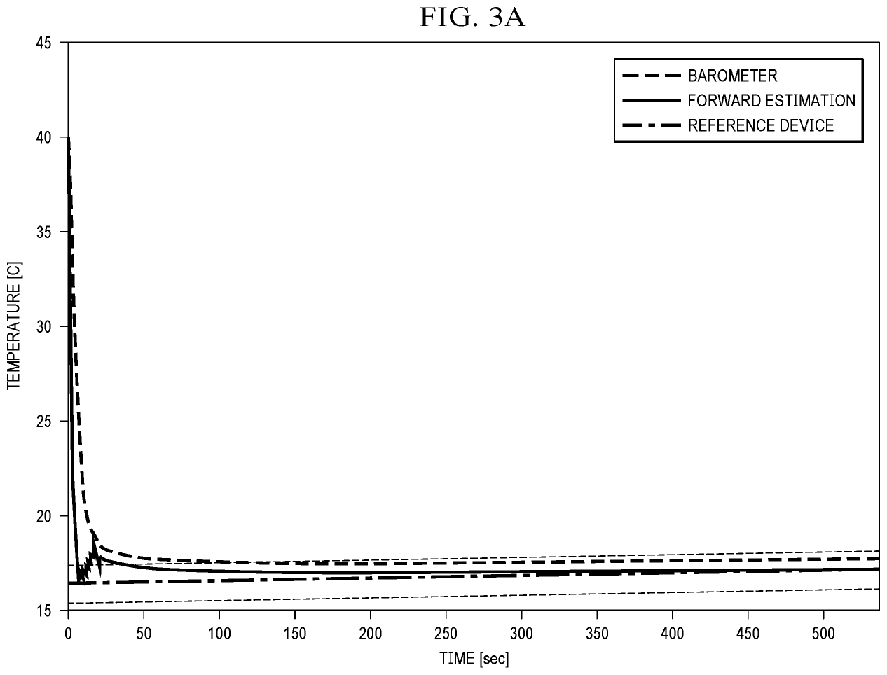

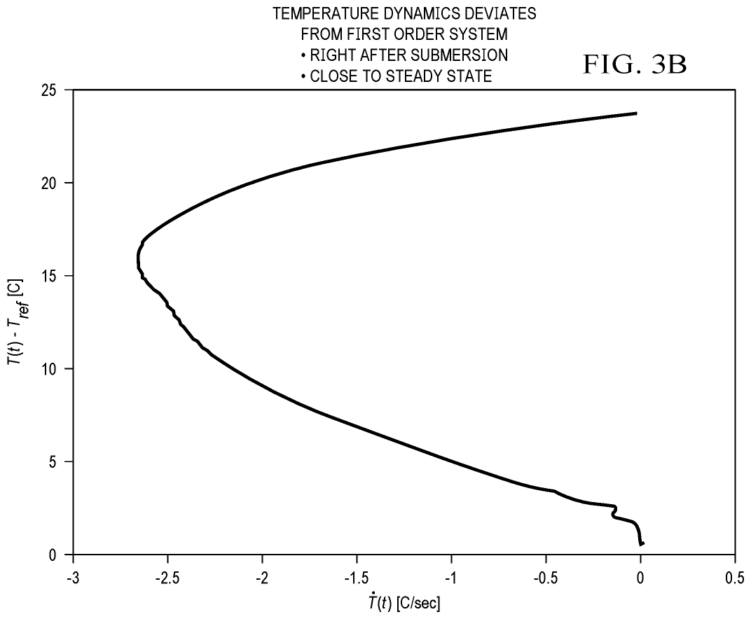

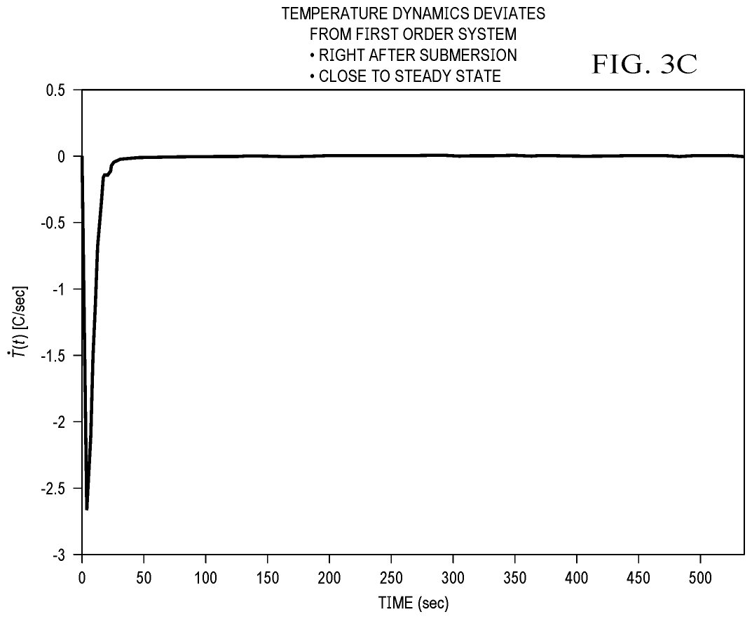

Note that the model of Equation [1] can be augmented with any desired number of higher order terms to take into account temperature readings from temperature sensors of the wearable device. In some embodiments, an offset term accounting for external thermal disturbances (e.g., sunlight, skin temperature) can be added to Equation [1] after a particular duration to remove a steady state bias shown in FIG. 3A. In some embodiments, internal components that generate thermal disturbances can be turned off (e.g., Wi-Fi radio, RF radio) while the water temperature is being estimated. In some embodiments, other states or variables can be modeled using machine learning, such as the thermal states of heat generating internal components, including but not limited to computer processors, RF transmitters/receivers (e.g., Wi-Fi, Bluetooth, GPS), battery heat dissipation, etc.

In some embodiments, the model in Equation [1] can be replaced with other data driven machine learning models, using for example other regression models (e.g., logistic regression), support vector machines, neural networks, decision trees and the like.

FIG. 3A is a graph illustrating temperature (Celsius) as a function of time, according to one or more embodiments. Temperature computed from raw temperature measured by the barometer, forward temperature estimation based on Equation [1] and reference temperature from a temperature sensor are shown. Based on this graph, one can observe that the raw temperature measured by the barometer takes over 2 minutes to reach thermal equilibrium with the water, and the forward temperature estimate based on Equation [1] takes about 10 seconds. Accordingly, the forward prediction model provides an accurate estimate of water temperature (e.g., +/−1° C. accuracy/resolution) much faster than the time needed for the raw temperature to reach equilibrium with the water. Also note in FIG. 3A the aforementioned steady state bias due to internal components in wearable device 100 having different times to reach thermal equilibrium with the water.

FIGS. 3B and 3C illustrate an example thermal model, according to one or more embodiments. Referring to FIG. 3B, the vertical axis is ΔT (the difference between pressure sensor temperature and temperature measured by a calibration device (i.e., reference temperature)) and the horizontal axis is the rate of change of temperature \dot{T}(t), which is shown in FIG. 3C. It is observed from FIG. 3B that there is a linear relationship for a portion of the curve between the rate of change of temperature and temperature error.

The data driven model of the relationship between the rate of change of temperature, \dot{T}(t), and temperature error, ΔT, is applied to the measured temperature data T(t) that is collected over an N second window (e.g., N=10 seconds). The result is the water temperature estimate \hat{T}_{water} is sped up compared to the time it would take for the pressure sensor to reach thermal equilibrium with the water. This relationship is modeled mathematically by the first order system in Equation [1]. In some embodiments, the data points for the ΔT versus \dot{T}(t) curve are included in a look-up table stored in memory of wearable device 100. In some embodiments, interpolation can be used to estimate points on the curve that fall between the points included in the look-up table.

### Submersion Detection

Water contact sensors used in some wearable devices may falsely identify wet devices as submerged. For example, if a user is taking a shower with the device, the wet device may falsely detect submersion. Accordingly, triggering automated actions based on submersion requires a detection method that is robust to a wide variety of conditions encountered by a wearable device.

In some embodiments, data from a barometer, accelerometer and gyroscope are used to measure the density of the surrounding medium (i.e., air or water) when a wearable device is moved vertically during normal user behavior. For example, ambient air pressure above the surface of the water is periodically measured to enable submersion detection at a minimum depth to further reduce false positives due to partial submersion. In some embodiments, a variety of additional sensors (e.g., a capacitive touch screen) with sensitivity to water provide a prior likelihood that wearable device 100 is wet to improve robustness and reduce latency of submerged/de-submerged detection. In some embodiments, RF radios (e.g., Wi-Fi, Bluetooth, GPS) embedded in wearable device 100 are used to reduce false positive submersion events. Because high frequency RF waves do not generally penetrate below the water surface, receiving a RF signal by a wireless receiver embedded in wearable device 100 is an indication of a false positive submersion event.

### Submerged/De-Submerged Classifier

In some embodiments, a classifier is used to determine when wearable device 100 is submerged in water or de-submerged. The classifier is based on the insight that, in water, large bursts in vertical acceleration are accompanied by a large variation in pressure due to the high density of water. In contrast, large bursts in vertical acceleration “in air” should correlated with relatively small pressure variation due to low air density.

Mathematically, a column of medium of density ρ and vertical length d in the Earth's gravitational field (g) exerts a pressure (P) given by:

P=ρ·g·d. [2]

Differentiating P twice gives:

$$\ddot{P} = ρ \cdot g \cdot {a}_{z \_ pressure} , \qquad [ 3 ]$$

$${a}_{z \_ pressure} = \frac{\ddot{P}}{ρ \cdot g} . \qquad [ 4 ]$$

Accordingly, vertical acceleration, α_{z}, measured by a motion sensor (e.g., an IMU), can be compared to vertical acceleration computed from pressure, α_{z_pressure}, obtained from a pressure sensor (e.g., a barometer) to classify water submersion/de-submersion. Because the ratio between pressure and acceleration is about 1000 times greater in water, comparing α_{z} and α_{z} pressure provides robust submerged/de-submerged detection. In some embodiments, IMU vertical acceleration, α_{z}, is measured in a Cartesian reference coordinate frame centered on an estimated gravity vector computed from acceleration and rotation rate output by an accelerometer and gyro sensor, respectively, that are embedded in wearable device 100.

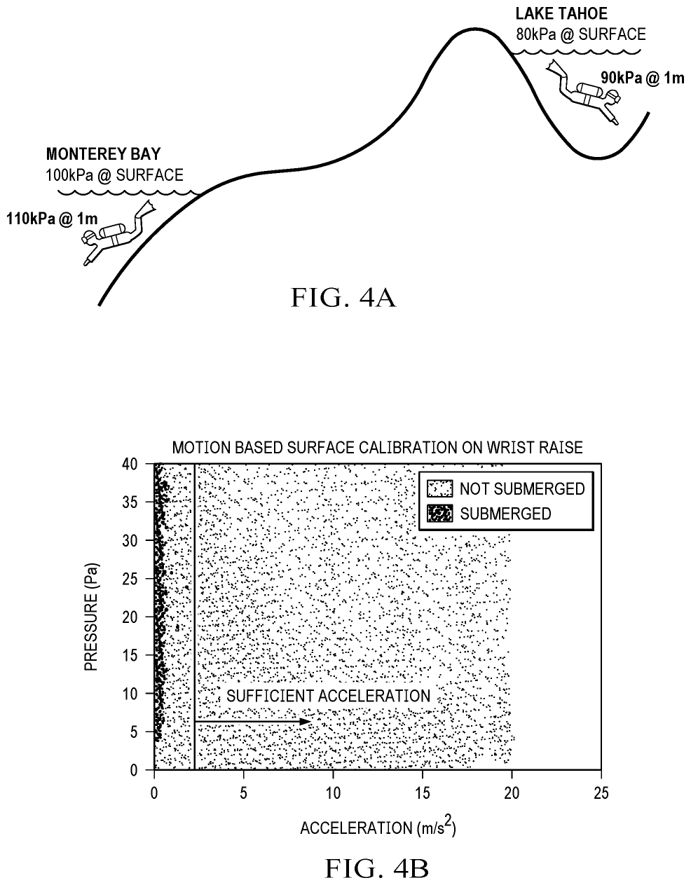

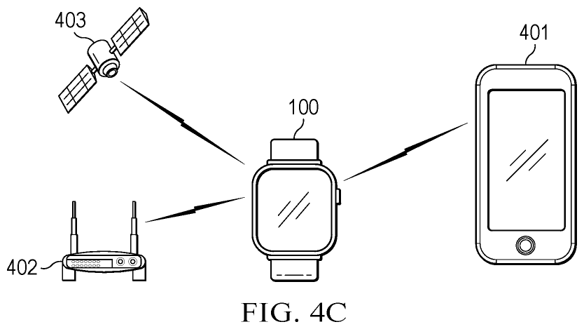

FIG. 4A illustrates the difference of surface pressure at different altitudes. In the example shown, the pressure is 100 kPa at the surface of Monterey Bay, California USA and 110 kPa at one meter below the surface. By contrast, the pressure is 80 kPa at the surface of Lake Tahoe, California USA, and 90 kPa at one meter below the surface. Estimating depth under water requires subtracting the barometric pressure at the surface, as described in Equation [5]:

water depth=(P_{baro}−P_{surface})/(ρ_{water}·g), [5 ]

P_{baro} is barometric pressure underwater, P_{surface} is the barometric pressure at the surface, ρ_{water} is water density and g is gravity.

As shown in Equation [5], the accuracy of the water depth calculation is dependent on an accurate P_{surface} measurement. However, as illustrated in FIG. 4A, P_{surface} is dynamic and depends on the altitude of wearable device 100. To obtain an accurate P_{surface} measurement, P_{surface} is measured each time the IMU vertical acceleration is above a specified minimum acceleration threshold and the range of measured pressure change is less than or equal to a specified threshold.

FIG. 4B is a graph of pressure (Pa) versus acceleration (m/s²) illustrating motion-based surface pressure calibration on wrist raise, according to one or more embodiments. If there is sufficient IMU vertical acceleration (e.g., α_{z}>3 m/s²) measured when the user raises her arm and the range of measured pressure change is less than or equal to 40 Pa, wearable device 100 is assumed to be not submerged; otherwise, is assumed that wearable device 100 may be submerged.

FIG. 4C illustrates radio-based surface pressure calibration, according one or more embodiments. Since RF signals cannot generally penetrate the water surface by more than a few centimeters, the signal reception by one or more RF radios embedded in wearable device 100 is used to trigger surface pressure calibration in addition to triggering based on IMU vertical acceleration. For example, if there is radio signal reception by a wireless transceiver embedded in wearable device 100 which was transmitted by smartphone 401, network router 402 or GPS satellites 403, it is assumed that wearable device 100 is “in air”, and surface pressure calibration is triggered.

FIG. 5A is a block diagram of a system for submersion detection and underwater depth estimation, according to one or more embodiments. System 500 includes accelerometer 501 (e.g., 3-axis accelerometer), gyroscope 502 (e.g., 3-axis gyroscope), which collectively is also referred to herein as an inertial measurement unit (IMU) or “motion sensors”), inertial vertical acceleration estimator 503, pressure sensor 504 (e.g., barometric pressure sensor), filtering and differentiation 505, pressure based vertical acceleration estimator 506, ambient density estimator 507, submerged/de-submerged classifier 508, RF radios 509, touch screen 510, electrocardiogram (ECG) sensor 511, depth estimator 512 and surface pressure calibrator 513.

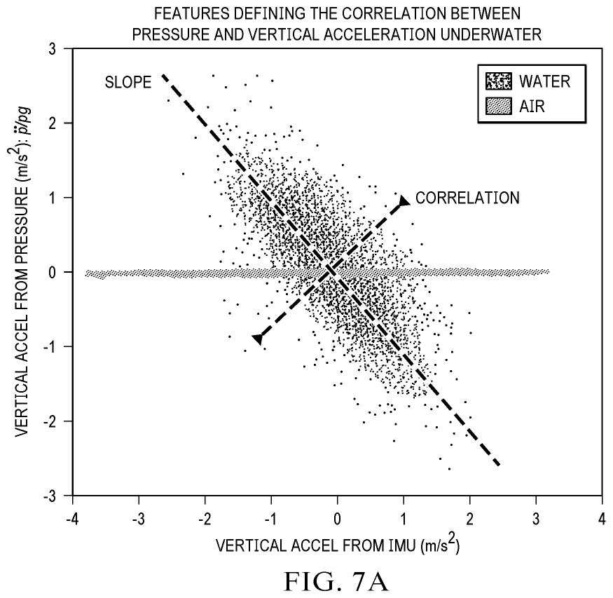

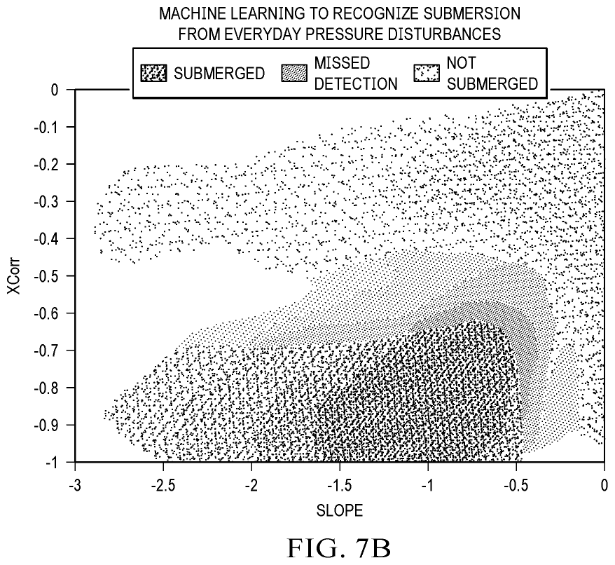

As previously described, inertial vertical acceleration estimator 503 estimates α_{z} in a reference coordinate frame centered on an estimated gravity vector using acceleration data and rotation rate data output by accelerometer 501 and gyroscope 502, respectively. Pressure sensor 504 outputs pressure data which is low-pass filtered and differentiated 505 to remove outliers and to provide differentiated pressure \ddot{P} from which pressure-based vertical acceleration estimator 506 computes pressure-based vertical acceleration, α_{z_pressure}, based on Equation [4]. The accelerations are stored in a buffer (e.g., store 1.5 seconds of acceleration data) so that ambient density estimator 507 can compute correlation and slope features based on the buffered accelerations, as described in reference to FIGS. 7A and 7B. The features are input into submerged/de-submerged classifier 508 (e.g., a support vector machine, neural network, etc.), which predicts one of a submerged class or not submerged class based on the input features.

In the lower branch of system 500, pressure data, P_{baro}, output from pressure sensor 504 is input into depth estimator 512 together with P_{surface} from surface calibration 513. Depth estimator 512 provides an estimated depth to submerged classifier 508 in accordance with Equation [6]. The estimated depth is to ensure that a minimum depth (e.g., 5 cm) is detected as a gate to performing the classification step.

In some embodiments, submerged/de-submerged classifier 508 also takes as input data from RF radios 509, touch screen 510 and ECG sensor 511. For example, wearable device 100 includes a projective capacitance touch screen that comprises a grid of small electric capacitors sensitive to detect variation in electric capacitance caused by touch of a human finger. The human body consists mostly of water. Water droplets on the touch screen can cause false positive touch detection. Water covering the touch screen (in the case of submersion) creates a signal indistinguishable from a full palm cover gesture that is detectable by the touch screen. Therefore, in some embodiments, detection of a full palm cover gesture feature can be an additional feature input into submerged/de-submerged classifier 508 to create a more robust classifier. In an embodiment, a prior signal generated by a full palm cover gesture is used to confirm the predicted class output by submerged/de-submerged classifier 508. In another embodiment, a submerged event can be verified by the presence or absence of a full palm cover gesture.

In some embodiments, wearable device 100 is a smartwatch that includes ECG sensor 511 to check for atrial fibrillation; a form of irregular heart rhythm. ECG sensor 511 has two electrodes: a first electrode is embedded into a back crystal module of the smartwatch, and the other electrode is attached to the watch crown. In other embodiments, the electrodes can be in other locations on the smartwatch. If the ECG sensor 511 is activated while the smartwatch is submerged into water, the electrodes produce an electrical “short” signal due to water electrical conductivity. In some embodiments, this “short” signal can be input into submerged/de-submerged classifier 508 and/or used to verify or rule out the predicted output of submerged/de-submerged classifier 508.

FIG. 5B is a block diagram of a process performed by surface pressure calibrator 513 of system 500 shown in FIG. 5A, according to one or more embodiments. Vertical pressure gradient estimator 521 estimates a vertical pressure gradient (change in pressure over unit distance) based on acceleration α_{z} from accelerometer 501 and pressure P_{baro} from pressure sensor 504, respectively. The vertical pressure gradient is input into local surface pressure estimator 513 together with radio data. In some embodiments, the local surface pressure estimate, P_{surface}, is given by:

P_{surface}=Δh˜ρₐᵢᵣ·g−P_{baro}, [6]

where Δₕ is the height above the water surface.

The local surface pressure, P_{surface}, computed by local surface pressure estimator 513 is input into pressure quality filter 517, which filters out outlier surface pressures based on “wet” activity history 516 (e.g., whether or not the barometric sensor was previously exposed to water) and outputs a calibrated local surface pressure, P_{surface}, based on the local surface pressure estimate and radio signal. The local surface pressure is stored in surface pressure calibration database 522, where it can be retrieved by depth estimator 512 to compute a water depth estimate, according to Equation [5].

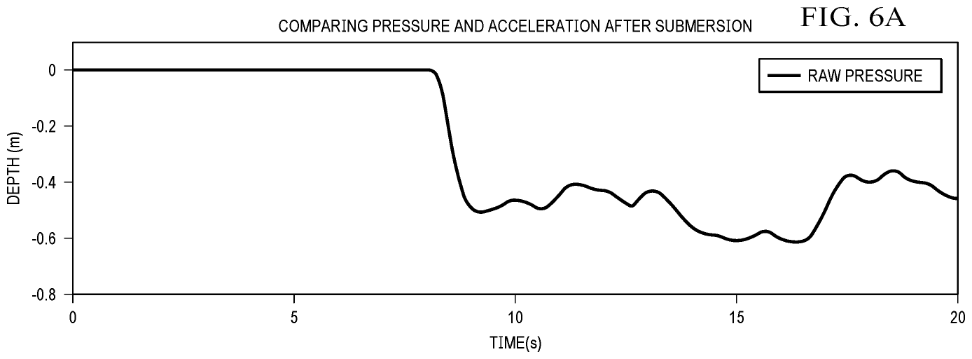

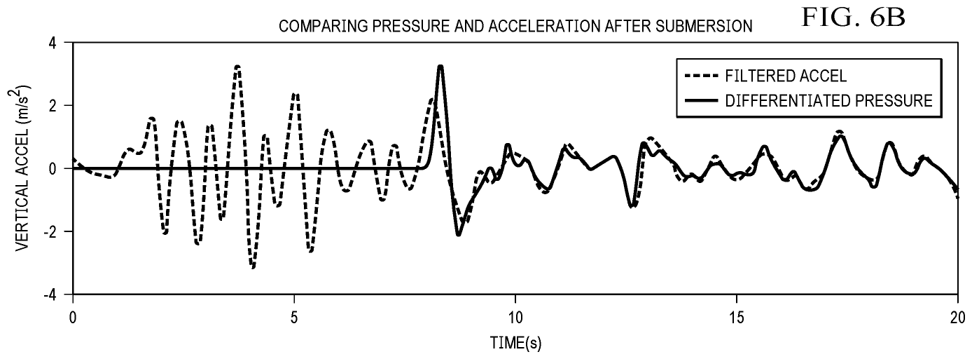

FIG. 6A is a graph of depth versus time before and after submersion, according to one or more embodiments. FIG. 6B is a graph of vertical acceleration versus time showing the tracking of filtered acceleration with differentiated pressure before and after submersion, according to one or more embodiments. As can be observed in FIG. 6B, the filtered IMU vertical acceleration, α_{z}, and differentiated pressure, \ddot{P}, are uncorrelated prior to submersion and then become highly correlated after submersion. This suggests that correlation between IMU inertial vertical acceleration and pressure can be used as a feature input to a classifier to detect submerged/de-submerged classes, as described more fully in reference to FIGS. 7A and 7B.

FIG. 7A is a graph of vertical acceleration from pressure versus vertical acceleration from the IMU, according to one or more embodiments. Data points for air and water are shown. Correlation and slope features defining the correlation between pressure and vertical acceleration underwater are labeled. FIG. 7B is a graph of correlation versus slope based on training data for different submerged and not submerged scenarios and missed detections, according to one or more embodiments.

In some embodiments, submerged/de-submerged classifier 508 can be a machine learning model (e.g., a support vector machine, neural network) that takes as features the correlation and slope shown in FIG. 7A and predicts one of two classes: “in air” and “in water.” Classifier 508 can be trained to recognize submersion from everyday pressure disturbances (e.g., closing doors, elevators, escalators). In some embodiments, classifier 508 can be trained on other features such a full cover palm gesture to address, e.g., showering scenarios while wearing the wearable device. In some embodiments, minimum and maximum values of vertical acceleration derived from pressure and from IMU are input into classifier 508 to address the scenario where, e.g., the wearable device is resting on a table.

### Example Processes

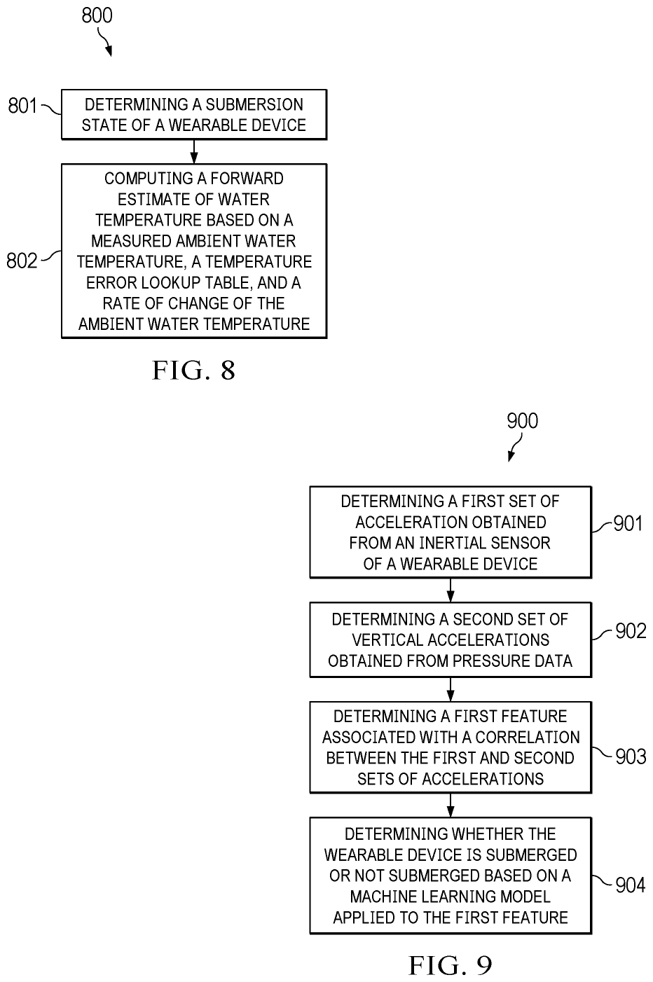

FIG. 8 is a flow diagram of a process 800 for temperature estimation, as described in reference to FIGS. 1-7.

Process 800 includes determining a submersion state of a wearable device (801), and responsive to the submersion state being submerged, computing a forward estimate of water temperature based on a measured ambient water temperature, a temperature error lookup table, and a rate of change of the ambient water temperature (802).

FIG. 9 is a flow diagram of a process 900 for submersion/de-submersion detection, as described in reference to FIGS. 1-7.

Process 900 includes determining a first set of vertical accelerations obtained from an inertial sensor of a wearable device (901), determining, with the at least one processor, a second set of vertical accelerations obtained from pressure data (902), determining a first feature associated with a correlation between the first and second sets of vertical accelerations (903), and determining whether the wearable device is submerged or not submerged in water based on a machine learning model applied to the first feature (904).

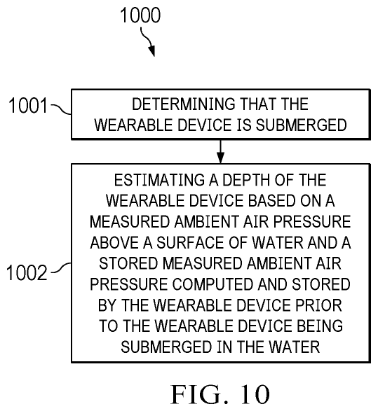

FIG. 10 is a flow diagram of a process 1000 for depth estimation, as described in reference to FIGS. 1-7.

Process 1000 includes determining that the wearable device is submerged (1001), estimating a depth of the wearable device based on a measured ambient pressure underwater and a stored measured ambient air pressure computed and stored by the wearable device prior to the wearable device being submerged in the water (1002).

### Example Device Architecture

FIG. 11 is a block diagram of a device architecture for implementing the features and processes described in reference to FIGS. 1-10. Architecture 1100 can include memory interface 1102, one or more hardware data processors, image processors and/or processors 1104 and peripherals interface 1106. Memory interface 1102, one or more processors 1104 and/or peripherals interface 1106 can be separate components or can be integrated in one or more integrated circuits. System architecture 1100 can be included in any suitable electronic device for crash detection, including but not limited to: a smartwatch, smartphone, fitness band and any other device that can be attached, worn, or held by a user.

Sensors, devices, and subsystems can be coupled to peripherals interface 1106 to provide multiple functionalities. For example, one or more motion sensors 1110, light sensor 1112 and proximity sensor 1114 can be coupled to peripherals interface 1106 to facilitate motion sensing (e.g., acceleration, rotation rates), lighting and proximity functions of the wearable device. Location processor 1115 can be connected to peripherals interface 1106 to provide geo-positioning. In some implementations, location processor 1115 can be a GNSS receiver, such as the Global Positioning System (GPS) receiver. Electronic magnetometer 1116 (e.g., an integrated circuit chip) can also be connected to peripherals interface 1106 to provide data that can be used to determine the direction of magnetic North. Electronic magnetometer 1116 can provide data to an electronic compass application. Motion sensor(s) 1110 can include one or more accelerometers and/or gyros configured to determine change of speed and direction of movement. Barometer 1117 can be configured to measure atmospheric pressure (e.g., pressure change inside a vehicle). Bio signal sensor 1120 can be one or more of a PPG sensor, an electroencephalogram (EEG) sensor, an electrocardiogram (ECG) sensor, an electromyogram (EMG) sensor, a mechanomyogram (MMG) sensor (e.g., piezo resistive sensor) for measuring muscle activity/contractions, an electrooculography (EOG) sensor, a galvanic skin response (GSR) sensor, a magnetoencephalogram (MEG) sensor and/or other suitable sensor(s) configured to measure bio signals.

Communication functions can be facilitated through wireless communication subsystems 1124, which can include radio frequency (RF) receivers and transmitters (or transceivers) and/or optical (e.g., infrared) receivers and transmitters. The specific design and implementation of the communication subsystem 1124 can depend on the communication network(s) over which a mobile device is intended to operate. For example, architecture 1100 can include communication subsystems 1124 designed to operate over a GSM network, a GPRS network, an EDGE network, a Wi-Fi™ network and a Bluetooth™ network. In particular, the wireless communication subsystems 1124 can include hosting protocols, such that the crash device can be configured as a base station for other wireless devices.

Audio subsystem 1126 can be coupled to a speaker 1128 and a microphone 1130 to facilitate voice-enabled functions, such as voice recognition, voice replication, digital recording, and telephony functions. Audio subsystem 1126 can be configured to receive voice commands from the user. Audio subsystem 1126 can be used to capture audio during a crash and to convert the audio to SPL for crash detection processing.

I/O subsystem 1140 can include touch surface controller 1142 and/or other input controller(s) 1144. Touch surface controller 1142 can be coupled to a touch surface 1146. Touch surface 1146 and touch surface controller 1142 can, for example, detect contact and movement or break thereof using any of a plurality of touch sensitivity technologies, including but not limited to capacitive, resistive, infrared and surface acoustic wave technologies, as well as other proximity sensor arrays or other elements for determining one or more points of contact with touch surface 1146. Touch surface 1146 can include, for example, a touch screen or the digital crown of a smart watch. I/O subsystem 1140 can include a haptic engine or device for providing haptic feedback (e.g., vibration) in response to commands from processor 1104. In an embodiment, touch surface 1146 can be a pressure-sensitive surface.

Other input controller(s) 1144 can be coupled to other input/control devices 1148, such as one or more buttons, rocker switches, thumbwheel, infrared port, and USB port. The one or more buttons (not shown) can include an up/down button for volume control of speaker 1128 and/or microphone 1130. Touch surface 1146 or other controllers 1144 (e.g., a button) can include, or be coupled to, fingerprint identification circuitry for use with a fingerprint authentication application to authenticate a user based on their fingerprint(s).

In one implementation, a pressing of the button for a first duration may disengage a lock of the touch surface 1146; and a pressing of the button for a second duration that is longer than the first duration may turn power to the mobile device on or off. The user may be able to customize a functionality of one or more of the buttons. The touch surface 1146 can, for example, also be used to implement virtual or soft buttons.

In some implementations, the mobile device can present recorded audio and/or video files, such as MP3, AAC and MPEG files. In some implementations, the mobile device can include the functionality of an MP3 player. Other input/output and control devices can also be used.

Memory interface 1102 can be coupled to memory 1150. Memory 1150 can include high-speed random-access memory and/or non-volatile memory, such as one or more magnetic disk storage devices, one or more optical storage devices and/or flash memory (e.g., NAND, NOR). Memory 1150 can store operating system 1152, such as the iOS operating system developed by Apple Inc. of Cupertino, California. Operating system 1152 may include instructions for handling basic system services and for performing hardware dependent tasks. In some implementations, operating system 1152 can include a kernel (e.g., UNIX kernel).

Memory 1150 may also store communication instructions 1154 to facilitate communicating with one or more additional devices, one or more computers and/or one or more servers, such as, for example, instructions for implementing a software stack for wired or wireless communications with other devices. Memory 1150 may include graphical user interface instructions 1156 to facilitate graphic user interface processing; sensor processing instructions 1158 to facilitate sensor-related processing and functions; phone instructions 1160 to facilitate phone-related processes and functions; electronic messaging instructions 1162 to facilitate electronic-messaging related processes and functions; web browsing instructions 1164 to facilitate web browsing-related processes and functions; media processing instructions 1166 to facilitate media processing-related processes and functions; GNSS/Location instructions 1168 to facilitate generic GNSS and location-related processes and instructions; and water temperature and depth estimation and submersion/de-submersion detection instructions 1170 that implement the processes described in reference to FIGS. 1-10. Memory 1150 further includes other application instructions 1172 including but not limited to instructions for applications that utilize estimated water temperature, water depth and submersion/de-submersion.

Each of the above identified instructions and applications can correspond to a set of instructions for performing one or more functions described above. These instructions need not be implemented as separate software programs, procedures, or modules. Memory 1150 can include additional instructions or fewer instructions. Furthermore, various functions of the mobile device may be implemented in hardware and/or in software, including in one or more signal processing and/or application specific integrated circuits.

While this specification contains many specific implementation details, these should not be construed as limitations on the scope of any inventions or of what may be claimed, but rather as descriptions of features specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable sub combination. Moreover, although features may be described above as acting in certain combinations and even initially claimed as such, one or more features from a claimed combination can in some cases be excised from the combination, and the claimed combination may be directed to a sub combination or variation of a sub combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

As described above, some aspects of the subject matter of this specification include gathering and use of data available from various sources to improve services a mobile device can provide to a user. The present disclosure contemplates that in some instances, this gathered data may identify a particular location or an address based on device usage. Such personal information data can include location-based data, addresses, subscriber account identifiers, or other identifying information.

The present disclosure further contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. For example, personal information from users should be collected for legitimate and reasonable uses of the entity and not shared or sold outside of those legitimate uses. Further, such collection should occur only after receiving the informed consent of the users. Additionally, such entities would take any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices.

In the case of advertisement delivery services, the present disclosure also contemplates embodiments in which users selectively block the use of, or access to, personal information data. That is, the present disclosure contemplates that hardware and/or software elements can be provided to prevent or block access to such personal information data. For example, in the case of advertisement delivery services, the present technology can be configured to allow users to select to “opt in” or “opt out” of participation in the collection of personal information data during registration for services.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, content can be selected and delivered to users by inferring preferences based on non-personal information data or a bare minimum amount of personal information, such as the content being requested by the device associated with a user, other non-personal information available to the content delivery services, or publicly available information.

What is claimed is:

1. A method comprising:
determining, with at least one processor, a first set of vertical accelerations obtained from an inertial sensor of a wearable device;
determining, with the at least one processor, a second set of vertical accelerations obtained from pressure data;
determining, with the at least one processor, a first feature associated with a correlation between the first and second sets of vertical accelerations; and
determining, with the at least one processor, whether the wearable device is submerged or not submerged in water based on a machine learning model applied to the first feature.

2. The method of claim 2, further comprising:
determining, with the at least one processor, a second feature associated with a slope of a line fitted to a plot of the first set of vertical accelerations and the second set of vertical accelerations; and
determining, with the at least one processor, that the wearable device is submerged or not submerged in the water based on the machine learning model applied to the first feature and the second feature.

3. The method of claim 2, further comprising:
determining, with the at least one processor, a second feature associated with a touch screen gesture; and
determining, with the at least one processor, whether the wearable device is submerged or not submerged in the water based the machine learning model applied to the first feature and the second feature.

4. The method of claim 1, wherein responsive to determining that the wearable device is submerged, the method further comprises:
estimating, with the at least one processor, a depth of the wearable device in the water based on a measured pressure and a measured ambient air pressure at the water surface computed and stored by the wearable device prior to the wearable device being submerged in the water.

5. The method of claim 4, wherein determining whether the wearable device is submerged or not submerged in the water further comprises:
comparing the estimated depth with a minimum depth threshold; and
if the estimated depth exceeds a minimum depth threshold, determining whether the wearable device is submerged or not submerged in water.

6. The method of claim 4, wherein the ambient air pressure at the surface is measured each time the first set of vertical accelerations are above a minimum threshold and a range of measured pressure change is less than or equal to a specified pressure threshold.

7. The method of claim 4, where the ambient air pressure is filtered to remove potential outliers due to a prior exposure of a pressure sensor of the wearable device to water.

8. A method comprising:
determining, with at least one processor, a water submersion state of a wearable device; and
responsive to the water submersion state being submerged, computing, with the at least one processor, a forward estimate of the water temperature based on a measured water temperature, a temperature error lookup table, and a rate of change of the ambient water temperature.

9. The method of claim 7, wherein the forward estimate of the water temperature is estimated by a first order forward temperature predictor given by:
\hat{T}_{water}=T(t)−ΔT(\dot{T}(t)),
where \hat{T}_{water} is the forward estimate of water temperature, T (t) is the measured ambient water temperature at time t, ΔT is a temperature error from a pre-established lookup table, as a function of \dot{T}(t), and \dot{T}(t) is the rate of change of temperature at time t.

10. An apparatus comprising:
at least one motion sensor;
at least one pressure sensor;
at least one processor;
memory storing instructions that when executed by the at least one processor, cause the at least one processor to perform operations comprising:
determining a first set of vertical accelerations obtained from the motion sensor;
determining a second set of vertical accelerations obtained from pressure data measured by the at least one pressure sensor;
determining a first feature associated with a correlation between the first and second sets of vertical accelerations; and
determining whether the apparatus is submerged or not submerged in water based on a machine learning model applied to the first feature.

11. The apparatus of claim 10, further comprising:
determining a second feature associated with a slope of a line fitted to a plot of the first set of vertical accelerations and the second set of vertical accelerations; and
determining that the apparatus is submerged or not submerged in the water based on the machine learning model applied to the first feature and the second feature.

12. The apparatus of claim 11, wherein the apparatus includes a touch screen, and the operations further comprise:
determining a second feature associated with a touch screen gesture; and
determining whether the apparatus is submerged or not submerged in the water based the machine learning model applied to the first feature and the second feature.

13. The apparatus of claim 10, wherein responsive to determining that the apparatus is submerged, the method further comprises:
estimating a depth of the apparatus in the water based on a measured pressure and a measured ambient air pressure at the water surface computed and stored by the apparatus prior to the apparatus being submerged in the water.

14. The apparatus of claim 13, wherein determining whether the apparatus is submerged or not submerged in the water further comprises:
comparing the estimated depth with a minimum depth threshold; and
if the estimated depth exceeds a minimum depth threshold, determining whether the apparatus is submerged or not submerged in water.

15. The apparatus of claim 13, wherein the ambient air pressure at the surface is measured each time the first set of vertical accelerations are above a minimum threshold and a range of measured pressure change is less than or equal to a specified pressure threshold.

16. The apparatus of claim 13, where the ambient air pressure is filtered to remove potential outliers due to a prior exposure of a pressure sensor of the wearable device to water.

17. An apparatus comprising:
at least one temperature sensor;
at least one processor;
memory storing instructions that when executed by the at least one processor, cause the at least one processor to perform operations comprising:
determining a water submersion state of a wearable device; and
responsive to the water submersion state being submerged, computing a forward estimate of the water temperature based on ambient water temperature measured by the at least one temperature sensor, a temperature error lookup table, and a rate of change of the ambient water temperature.

18. The apparatus of claim 17, wherein the forward estimate of the water temperature is estimated by a first order forward temperature predictor given by:
\hat{T}_{water}=T(t)−ΔT(\dot{T}(t)),
where \hat{T}_{water} is the forward estimate of water temperature, T(t) is the ambient air temperature measured by the at least one temperature sensor at the water surface at time t, ΔT is a temperature error from a pre-established lookup table as a function of \dot{T}(t), and \dot{T}(t) is the rate of change of temperature at time t.
